(19) United States
(12) Patent Application Publication
(10) Pub. No.: US 2019/0237199 A1
(43) Pub. Date: Aug. 1, 2019

(54) POPULATION CLASSIFICATION FOR CARE MANAGEMENT

(71) Applicant: International Business Machines Corporation, Armonk, NY (US)

(72) Inventors: Anne E. Fischer, Chelsea, MI (US); Robert P. Kelley, Ann Arbor, MI (US); Amy G. Brown, Carmel, IN (US); Janet K. Young, Ann Arbor, MI (US); George Sirbu, Ann Arbor, MI (US); Christopher A. Gagen, Ann Arbor, MI (US); Matthew R. Alter, Ann Arbor, MI (US); Therese R. Gorski, Ann Arbor, MI (US)

(21) Appl. No.: 15/883,831

(22) Filed: Jan. 30, 2018

(51) Int. Cl. G16H 50/70; G16H 10/60

(52) U.S. Cl. CPC G16H 50/70; G16H 10/60

(57) ABSTRACT

A method of population classification to identify the likely level of care management interventions appropriate for individuals, for use in a population health management system, the method including: receiving, by a processing engine, medical claims data for an individual; reviewing, by the processing engine, a plurality of predetermined parameters within the medical claims data for the individual; classifying, by the processing engine, the individual into one of a plurality of predetermined intervention groups, based on reviewing the plurality of predetermined parameters; and providing to the population health management system, by the processing engine, a level of care management appropriate for the individual based on the predetermined intervention group in which the individual was classified.

### BACKGROUND

The present disclosure relates to methods and systems for population classification to identify near term care management needs and supporting interventions for individuals for use in a population health management system.

While existing systems may classify individuals based on similar clinical conditions and/or anticipated resource use, these methods are not optimized to consider the level of care management interventions that would be most appropriate based on current needs of an individual.

### SUMMARY

According to a first aspect, the disclosure provides a method of population classification to help identify the likely level of care management intervention required, for use in a population health management system. The method includes receiving, by a processing engine, medical claims data for an individual; reviewing, by the processing engine, a plurality of predetermined parameters within the medical claims data for the individual; classifying, by the processing engine, the individual into one of a plurality of predetermined intervention groups based on reviewing the plurality of predetermined parameters; and providing to the population health management system, by the processing engine, care management level based on the predetermined intervention group in which the individual has been classified.

According to another aspect, the disclosure provides a system for population classification to identify the likely level of care management intervention required for individuals, for use in a population health management system. The system includes a processing engine that is configured to receive medical claims data for an individual; review a plurality of predetermined parameters within the medical claims data for the individual; classify the individual into one of a plurality of predetermined intervention groups based on reviewing the plurality of predetermined parameters; and provide to the population health management system, a care management level based on the predetermined intervention group in which the individual has been classified.

In yet another aspect, the disclosure provides a computer program product for population classification to identify the likely level of care management intervention required for individuals, for use in a population health management system. The computer program product includes a computer readable storage medium having program instructions embodied therewith whereby the program instructions are executable by a processing engine. The program instructions cause the processing engine to receive medical claims data for an individual; review a plurality of predetermined parameters within the medical claims data for the individual; classify the individual into one of a plurality of predetermined groups that suggest intervention levels, based on reviewing the plurality of predetermined parameters; and provide to the population health management system, a care management level for the individual based on the predetermined intervention group in which the individual has been assigned.

These and other aspects, objects, and features of the present disclosure will be understood and appreciated by those skilled in the art upon studying the following specification, claims, and appended drawings.

### BRIEF DESCRIPTION OF THE DRAWINGS

In the drawings:

FIG. 1 depicts a schematic representation of a diagram of the various intervention groups, according to an embodiment of the present disclosure;

FIG. 2 depicts an exemplary schematic representation of a network environment in which aspects of the present disclosure may be implemented;

FIGS. 3A-3D depict a flowchart of a method for population classification to identify the level of care management intervention appropriate for individuals, for use in a population health management system, according to an embodiment of the present disclosure; and

FIG. 4 depicts a chart and example of an individual transitioning between various intervention groups.

### DETAILED DESCRIPTION

The present disclosure provides a method and system for population classification to identify potential care management intervention levels for individuals, for use in a population health management system. More specifically, the system and method described herein provides a framework for putting individuals in categories where there are similarities in the type of care management strategy that may be taken.

The system utilizes a software implemented method to process data to calculate the recommended care management intervention level based on specific variables identified for each group. In doing so, the system creates variables that can be used in characterizing an individual's conditions and utilization. The system and method described herein provide more than a statistical identification of “high risk” or “high cost” individuals. The actionable intervention level groups provide insight on the entire population, including distribution of individuals needing various levels of care management. Thus, the method can help to improve population health while considering the level of care management intervention best suited for a given group of individuals. The method combines a data and knowledge driven approach and provides a framework for more specific targeted analytics.

The system and method are not provided to determine how a physician should treat a patient but allows for care managers to determine at what level an individual may need care management assistance. The method looks holistically at individuals as to the level of care management intervention they may require at that particular time. These intervention levels can change over time due to changes in an individual's medical conditions, healthcare utilization patterns, and other factors.

The intervention groups were created by examining one year of detailed utilization information from inpatient, outpatient and drug claims for a sample of individuals from MarketScan®, a proprietary database containing healthcare data from claims of individuals with employer based health insurance. The detailed claims for these individuals were reviewed, and then each person was assigned to a care management level intervention group. Variables, such as significant chronic disease count, count of maintenance drugs, ER visits in the last 3 months, number of specialists in the past 3 months, recent significant procedures, etc. were created, and utilized to generate a statistical model which predicts the most appropriate intervention group, using the assigned category based on expert review as the gold standard.

Before describing the method in more detail, a description of the system is first provided.

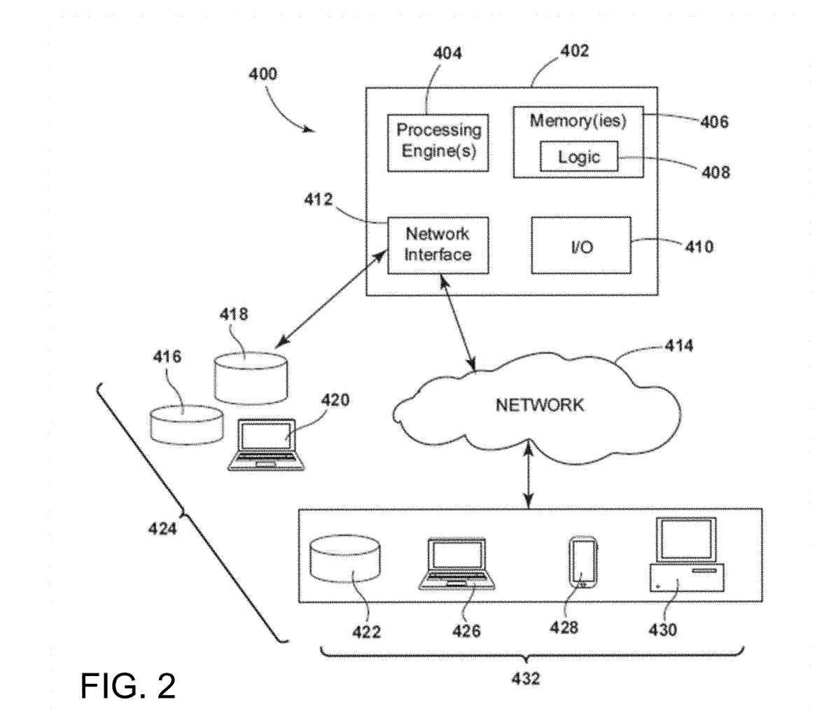

Referring to FIG. 2, a schematic representation of a system 400 for determining the population classification is shown. In accordance with an embodiment described herein, system 400 includes a processing engine 404, which can include one or more sub-engines. Processing engine 404 may be coupled to a population health management system 432. Processing engine 404 may also be coupled to one or more databases 416, 418, as well as other processing engines. The system 400 is explained in more detail below.

As described in more detail below, processing engine 404 may also include, or be coupled with, one or more database(s) to store information such as input variables and algorithms for implementing data processing rules. Accordingly, processing engine 404 may receive and process the input variables based on the data processing rules. Processing engine 404 may also be hosted on one or more servers including any processor, server (including a cloud server), mainframe computer, or other processor-based device capable of facilitating communication and running software programs or other applications.

Accordingly, aspects of the present disclosure provide methods, systems and computer program products for population classification to identify intervention levels for individuals, for use in a population health management system.

Embodiments of the methods and system described herein may utilize various computer software and hardware components including, but not limited to, servers, mainframes, desktop computers, databases, computer readable media, input/output devices, networking components and other components as would be known and understood by a person skilled in the art. FIG. 2 illustrates a networked operating system 400 in which aspects of the present disclosure may be implemented, according to embodiments described herein. It should be understood, however, that system 400 is only one example of a suitable environment for implementing methods described herein and is not intended to suggest any limitation as to the scope or functionality of the present disclosure. As depicted, system 400 may include one or more servers 402; one or more databases 416, 418, 420 and 422, collectively; databases 424; and one or more access devices, such as computer/laptop computer 426, handheld device 428 and enterprise device 430, collectively access devices 426, 428, and 430. Components of system 400 may also be communicatively connected to one or more networks, such as network 414, for communication between the components.

Server 402 is generally representative of one or more servers suitable for processing medical claims data and serving data in the form of webpages or other markup language forms with associated applets, ActiveX controls, remote-invocation objects, or other related software and data structures, to service clients of various “thicknesses.” Server 402 may be configured as would be known by a skilled artisan and may include one or more processing engines 404, memory 406, one or more network interfaces 412, one or more input/output devices 410 (such as a keyboard, mouse, display, etc.). Memory 406 may include a logic module 408 for processing medical claims data. In some embodiments, processing engine 404 may be hosted on one or more local or distributed processors, controllers, or virtual machines.

As would be understood in the art, processing engine 404 may be configured in any convenient or desirable form as would be known by a skilled artisan. Memory 406 may comprise one or more electronic, magnetic, or optical data-storage devices, and may include different types of memory. As would be known in the art, memory 406 may store instructions, such as logic module 408, for processing by processing engine 404. Logic module 408 may include machine readable and/or executable instructions sets for performing and/or facilitating performance of methods and rendering graphical or tabular user interfaces as further described herein, including sharing one or more portions of this functionality in a client-server architecture, over a wireless or wireline communications network 414 with one or more access devices 426, 428, and 430. The logic may be embodied in a variety of known software systems including, but not limited to, SPSS, Python, SAS® and Java®.

Databases 424 may include one or more electronic, magnetic, optical data-storage devices, or other data-storage devices which can include or are otherwise associated with respective indices (not shown). In some embodiments, databases 424 include medical, drug, and lab-related medical claims data. In other embodiments, databases 424 include and/or extract healthcare administrative data, such as medical claims and encounter data, from health plan, employer and government databases. In some embodiments, databases 424 additionally include medical guidelines data sources, such as government and/or other public sources, government regulations and proprietary databases. According to aspects described herein, databases 424 may be connected to server 402 via network 414.

Server 402 may be accessed by one or more access devices including, but not limited to, personal computers, enterprise workstations, handheld devices, mobile telephone, or any other device capable of providing an effective user interface with a server or database. As depicted, in an embodiment of the disclosure, server 402 is connected to one or more access devices 426, 428, and 430 via network 414. Network 414 may be any type of data communications network known in the art, including, but not limited to a LAN, WAN, public-switched, satellite, or any other type of network as would be contemplated by a skilled artisan.

Accordingly, the present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium (e.g., memory 406) can be a tangible device that can retain and store instructions (e.g., logic module 408) for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Java, Python, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

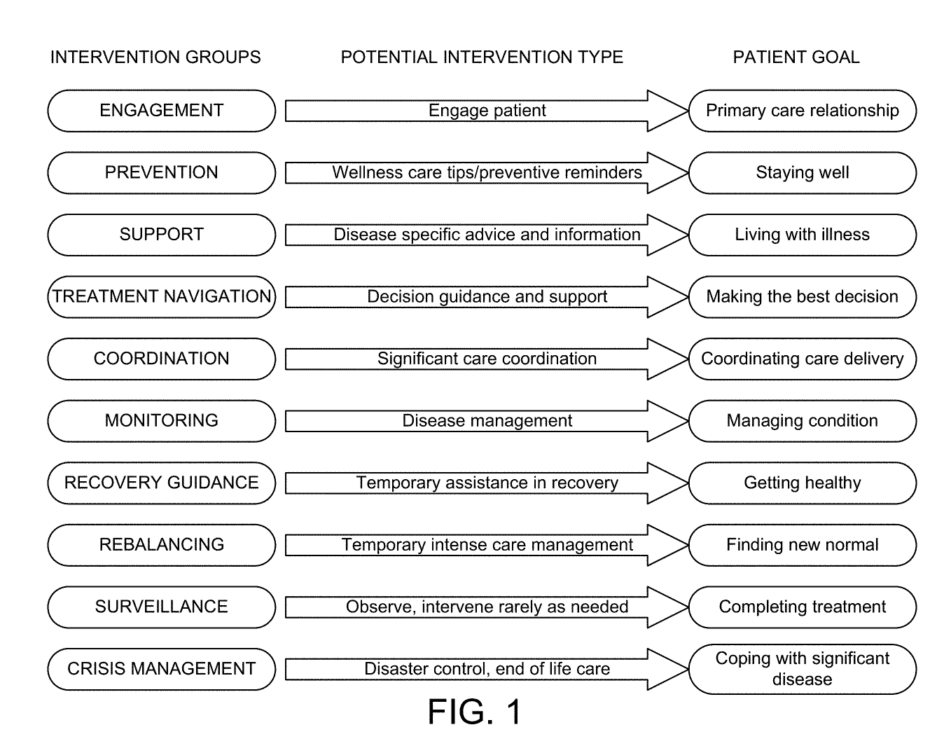

For purposes of example, 10 different care management intervention level groups are described herein. As shown in FIG. 1, these 10 intervention groups include Engagement, Prevention, Support, Treatment Navigation, Coordination, Monitoring, Recovery Guidance, Rebalancing, Surveillance, and Crisis Management. These intervention groups represent different levels of intervention and are assigned using a priority schema such that the highest ranked category is identified first for a given individual. In the example described herein, the intervention groups are listed in order in FIG. 1 with Engagement being the lowest priority for assignment and Crisis Management being the highest priority within the assignment hierarchy. As further described below, individuals are categorized into the highest priority group within the assignment hierarchy, for which they are most qualified based on the variables (also referred to herein as parameters). Note that the prioritization reflects the perceived most urgent situation for a given individual, not necessarily the highest level of care management intervention they might qualify for. For example, an individual in Crisis Management (the highest priority category) may not require a high level of care management intervention because they are already likely being managed by a team of physicians.

Each of the intervention groups suggest different levels of care management to obtain different goals. Thus, for example, in the Engagement intervention group, individuals typically do not have a primary care relationship with a specific physician. Individuals in the Engagement intervention group also typically do not have a lot of utilization history, with the possible exception of claims from urgent care or emergency facilities. Thus, a potential intervention may be to engage the individual with the goal of establishing a primary care relationship with a physician. This would be considered a lower level of care management.

The individuals categorized in the Prevention intervention group are typically healthy, although they may have one or a few minor conditions. Such minor conditions may be, for example, controlled hypertension or gastric reflux. A potential intervention for individuals in the Prevention intervention group may be to provide wellness care tips and preventive reminders with the goal of having the individual remain healthy. Again, this is a lower level of care management.

Individuals in the Support intervention group tend to have a more significant chronic condition, such as chronic obstructive pulmonary disease (COPD) or diabetes, but seem to be successfully managing their condition. For these individuals, the goal may be living with the illness and a potential care management action might be to provide disease specific advice and information.

Individuals in the Treatment Navigation intervention group are those who are newly diagnosed with a condition for which there is more than one clinically valid treatment option, and where there is often significant personal preference reflected in the decision of which option to pursue. For example, if an individual has osteoarthritis of their knee, they may have treatment options of physical therapy or arthroscopic surgery or yet other options for treatment. Here the goal is to help the individual understand their treatment options, so that they can make the best decision on which treatment options to pursue. The potential intervention is education surrounding options and decision guidance with the goal of allowing the individual to make an informed choice.

Individuals categorized in the Coordination intervention group are those with several different chronic conditions as well as those who are often taking several different medications and/or seeing a number of different physician specialist types. The role of care management in these individuals is to coordinate care delivery with the goal of a consistent treatment plan across all providers.

The Monitoring intervention group includes individuals whose pattern of utilization of medical services suggests a need for guidance in more appropriate use of resources or services.

For example, these individuals may use the emergency room for non-emergent care, or for issues related to chronic conditions that might be preventable through better use of primary care. Another group of individuals requiring monitoring are those with unusually high use of opioids. Thus, through care management intervention, those in the Monitoring intervention group may be guided to using resources more appropriately.

Individuals in the Recovery Guidance intervention group have a recent significant condition or procedure from which they are expected to fully recover. For example, they may have just had hernia surgery and are expected to fully recover. These individuals would have the goal of returning to their baseline and would require intervention in the form of temporary assistance during their recovery.

Individuals in the Rebalancing intervention group typically have recently received a new major diagnosis like diabetes, or a life-changing procedure like amputation, or had a major accident causing significant changes, such as paralysis. These individuals may have the goal of finding a new ‘normal’ and may need temporary intense care management to help them cope with this significant change in health status.

The Surveillance intervention group may include individuals who already have a close relationship with the healthcare system based upon ongoing treatment for a single serious condition. For example, this may include individuals who are undergoing active cancer treatment with frequent chemotherapy or radiation treatments. This may also include other individuals such as those who may have had a recent organ transplant or are undergoing dialysis on an ongoing basis. Insofar as these individuals are already very closely monitored and undergoing treatments, care management intervention is rarely needed so there may only be a need to observe while assisting the individual in completing treatment.

Individuals in the Crisis Management intervention group are those with extremely serious conditions that appear to be poorly controlled. Examples may include individuals with significant organ failure and/or who are becoming septic, etc. Here the care management intervention may be focused on preventing catastrophic events and/or assisting with end of life care arrangements with the goal of coping with life-threatening conditions(s).

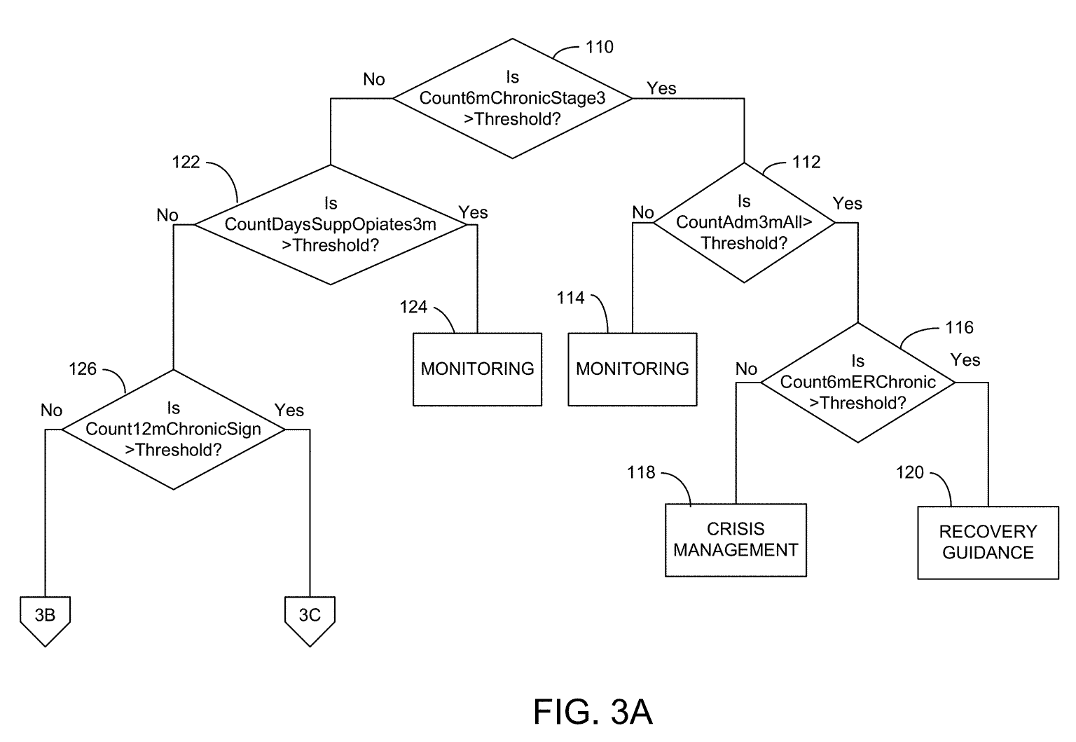

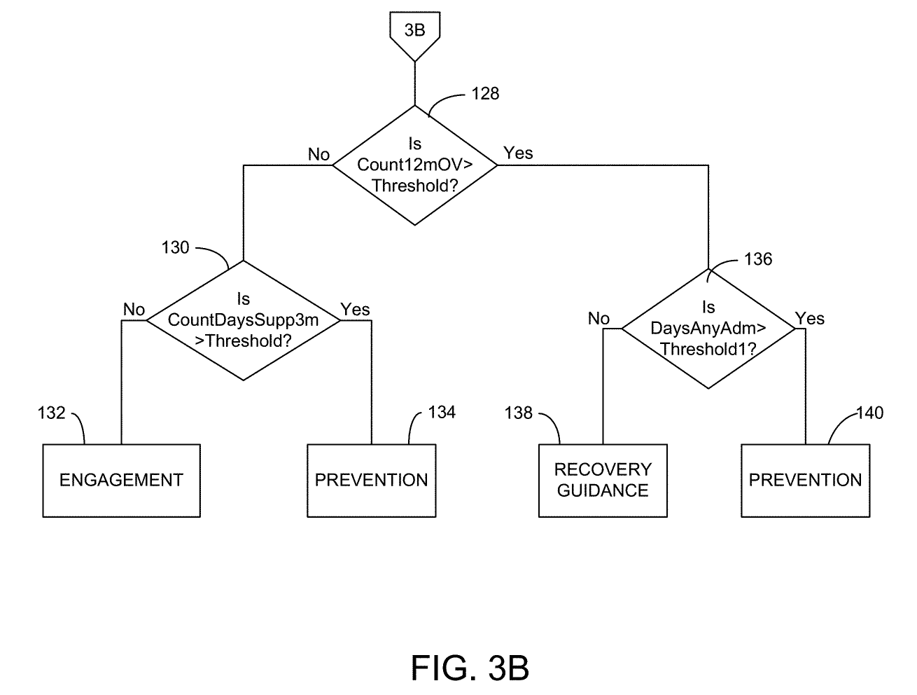

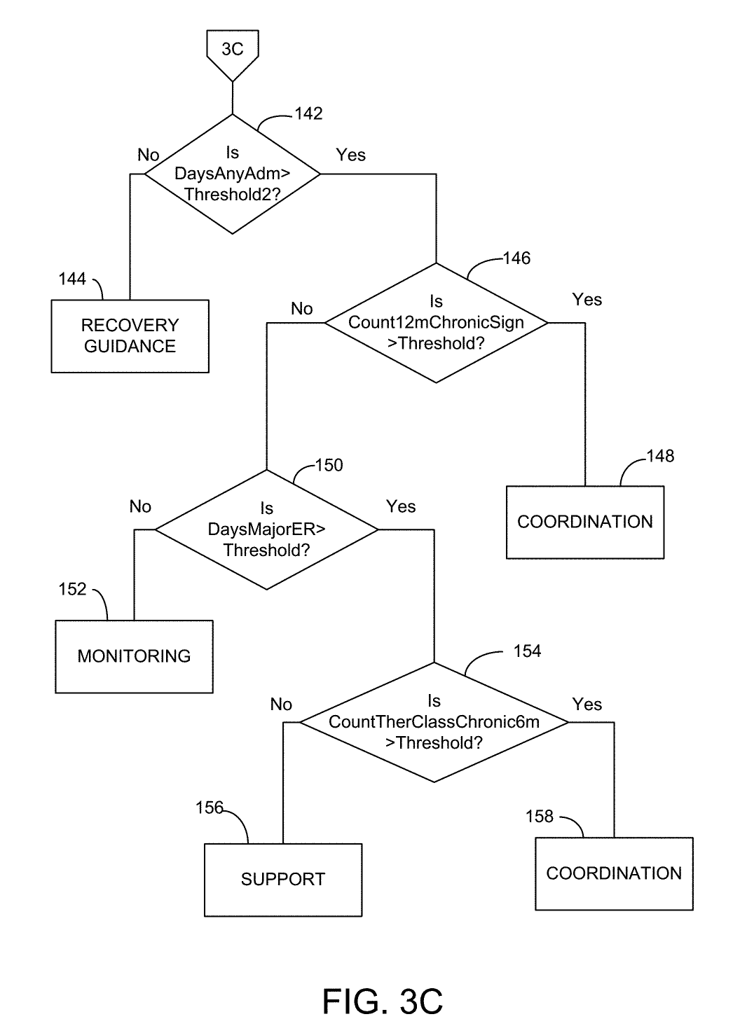

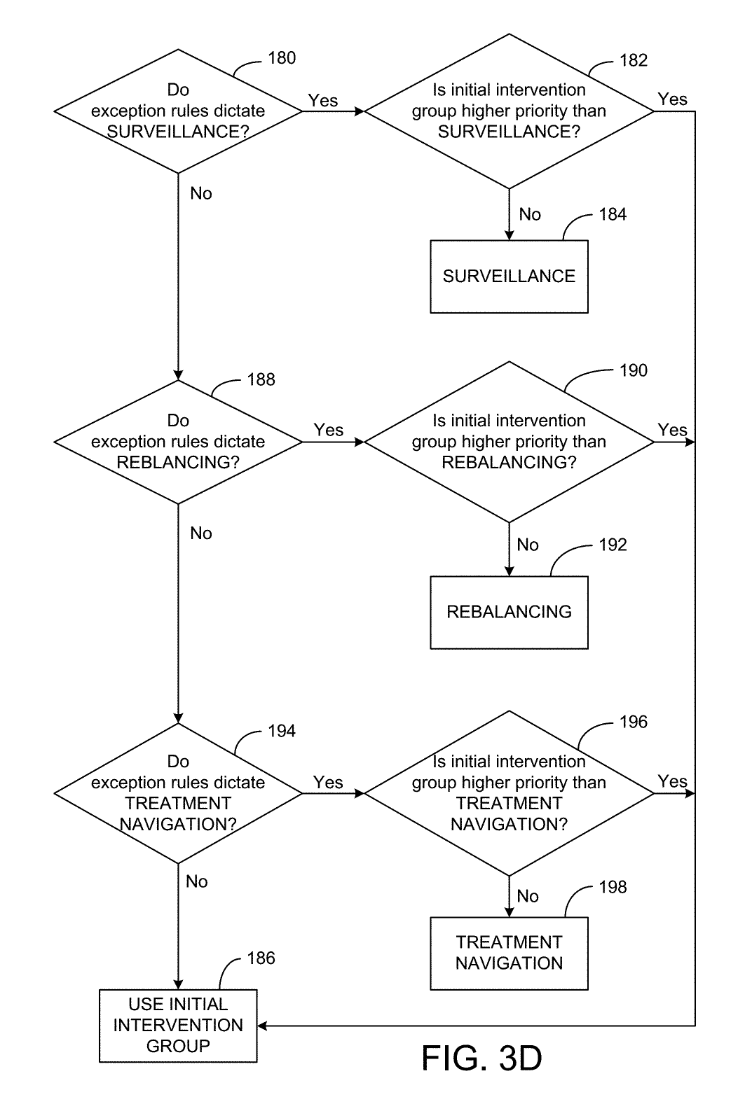

To categorize individuals into these intervention groups, various parameters may be analyzed. In the example described below, ten parameters are used to categorize individuals into one of seven of the intervention groups (all the groups with the exceptions of Treatment Navigation, Rebalancing, and Surveillance) using a decision tree represented by the flow chart in FIGS. 3A-3C. After the initial assignment to one of the seven intervention groups, rules are run to determine whether the individuals should be categorized into one of the Treatment Navigation, Rebalancing, and Surveillance intervention groups per FIG. 3D.

Prior to a description of the flowchart of FIGS. 3A-3D, a description is first provided of the parameters used in the flowchart to categorize the individuals into the initial seven intervention groups. These parameters are described as follows:
1. Days Since Inpatient Admission (DaysAnyAdm)—how many days since any prior hospital admission
2. Number of Admissions in Last 3 Months (CountAdm3mAll)
3. Count of Conditions that are Both Chronic and Significant Over the Last 12 Months (Count12mChronicSign) where conditions are identified using ICD-9-CM and ICD-10-CM diagnosis codes mapped to proprietary categories using Disease Staging
4. Count of Conditions that are Both Chronic and Stage 3 or Higher in the Past 6 Months (Count6mChronicStage3) where Conditions are identified as described above, and stage is identified by Disease Staging
5. Count of Emergency Room Visits for Chronic Conditions in the Past 6 Months (Count6mERChronic), where conditions are identified as described above
6. Days Since Last Major Emergency Room Visit (DaysMajorER) where major is defined based on Disease Staging categories and/or stages
7. Count of Office Visits Over the Past 12 Months (Count12mOV)
8. Sum of Days Supply of Prescription Medications Over the Past 3 Months (CountDaysSupp3m)
9. Sum of the Days Supply of Opiate Prescription Drugs Over the Past 3 Months (CountDaysSuppOpiates3m)
10. Count of Unique Intermediate Therapeutic Drug Classes With at Least One Chronic Drug Over the Past 6 Months (CountTherClassChronic6m) where therapeutic classes and chronic drugs are defined by REDBOOK®, a comprehensive resource for prescription and over-the-counter drug information developed and maintained by IBM Watson Health.

Although the ten parameters described above are used in the algorithm described below, different parameters, different thresholds for the parameters, and different numbers of such parameters may be used.

There are five selected grouping or classification methodologies used in the intervention group assignment. These include Disease Staging, Intermediate Therapeutic Drug Class defined by REDBOOK®, MS-DRG, and Procedure Groups.

Disease Staging (DS) produces output that is used in several variables that utilize information on chronicity, significance and/or severity of an individual's condition(s) as well as all 3 rule-based intervention group categories (Treatment Navigation, Rebalance, Surveillance). Disease Staging is a classification system that uses diagnoses to produce clusters of patients based on etiology, pathophysiology and severity. In this model, we use DS to classify each condition as chronic or acute, and whether or not the condition is highly significant, as well as the level of severity of a condition. Those classified as stage 3 typically involve several anatomic sites or include systemic complications, which are indicative of high severity. Stage 4 is death. Examples of DS categories that have been identified as significant include Cardiomyopathies, Ulcerative Colitis and Malaria.

Drug information is utilized in variables that consider counts or days supply of unique drugs or drug classes as well as 2 of the 3 rule based categories (Rebalancing and Surveillance). NDC is referenced in the diagnostic parameters above, which is the National Drug Code Directory of the Food and Drug Administration. NDC is rolled up to detailed Therapeutic Class; which is then rolled up to Intermediate Therapeutic Drug Class and then General Drug Class. NDC is also used to determine whether the drug is “chronic” (given mainly for chronic conditions) or “acute.” Examples of maintenance drugs include Fluoxetine, Metformin, and Atenolol.

MS-DRGs are included in logic to assign individuals to Surveillance or Rebalancing. MS-DRG is a Medicare Severity-Diagnosis Related Group, which uses diagnostic information, procedure information, gender, and discharge status for inpatients to assign individuals to mutually exclusive groups with similar expected costs.

Procedure Groups are used in logic to assign individuals to Surveillance or Rebalancing. Procedure Groups are a proprietary method to create rollup groups of CPT® and HCPCS codes into groups containing similar procedures. For example, CPT® codes 58260-58294, all of which are types of vaginal hysterectomy, are rolled up into a single procedure group.

With reference to FIGS. 3A-3D, the method is described, which is implemented using an algorithm executed by the processing engine 404. In the method, an initial intervention group is first assigned to an individual in FIGS. 3A-3C using the above parameters. Then, in FIG. 3D, exception rules are applied to determine if an exception to the initial intervention group should be made to assign a higher priority intervention group to the individual if certain conditions of the exception rules apply.

Referring first to FIG. 3A, processing engine 404 first determines whether the diagnostic parameter Count of Conditions that are Both Chronic and Stage 3 or Higher in the Past 6 Months (Count6mChronicStage3), which is the number of chronic stage 3 or higher conditions, based on DS, in the past 6 months, for the individual exceeds a threshold level in step 110. If this threshold is exceeded, the processing engine 404 proceeds to step 112. Otherwise, the processing engine 404 proceeds to step 122.

In step 112, the processing engine 404 determines whether the diagnostic parameter Number of Admissions in Last 3 Months (CountAdm3mAll), which is the number of hospital admissions for an individual over the last 3 months, exceeds a threshold. If CountAdm3mAll does not exceed the threshold, processing engine 404 initially assigns the individual to the Monitoring intervention group in step 114. Otherwise the processing engine 404 proceeds to step 116.

In step 116, the processing engine 404 determines whether the diagnostic parameter Count of Emergency Room Visits for Chronic Conditions in the Past 6 Months (Count6mERChronic), which is the number of ER visits for chronic conditions in the past 6 months, exceeds a threshold. If Count6mERChronic does not exceed this threshold, processing engine 404 initially assigns the individual to the Crisis Management intervention group in step 118. Otherwise the processing engine 404 initially assigns the individual to the Recovery Guidance intervention group in step 120.

In step 122, the processing engine 404 then determines whether the diagnostic parameter Sum of the Days Supply of Opiate Prescription Drugs Over the Past 3 Months (CountDaysSuppOpiates3m), which is the sum of the day's supply of opiates over the past 3 months, exceeds a threshold. If CountDaysSuppOpiates3m exceeds the threshold, processing engine 404 initially assigns the individual to the Monitoring intervention group in step 124. Otherwise the processing engine 404 proceeds to step 126.

In step 126, the processing engine 404 determines whether the diagnostic parameter Count of Conditions that are Both Chronic and Significant Over the Last 12 Months (Count12mChronicSign), which is a count of significant chronic conditions over the past 12 months, exceeds a threshold. If Count12mChronicSign does not exceed the threshold, the processing engine 404 proceeds to step 128 (FIG. 3B), otherwise it proceeds to step 142 (FIG. 3C).

Referring now to FIG. 3B, in step 128, the processing engine 404 determines whether the diagnostic parameter Count of Office Visits Over the Past 12 Months (Count12mOV), which is a count of office visits over the past 12 months, exceeds a threshold. If Count12mOV does not exceed the threshold, processing engine 404 proceeds to step 130, otherwise it proceeds to step 136.

In step 130, the processing engine 404 determines whether the diagnostic parameter Sum of Days Supply of Prescription Medications Over the Past 3 Months (CountDaysSupp3m), which is the day's supply of drugs over the past 3 months, exceeds a threshold. If CountDaysSupp3m is less than or equal to this threshold, processing engine 404 initially assigns the individual to the Engagement intervention group in step 132. Otherwise the processing engine 404 initially assigns the individual to the Prevention intervention group in step 134.

In step 136, the processing engine 404 determines whether the diagnostic parameter Days Since Inpatient Admission (DaysAnyAdm), which is the number of days since any hospital admission, exceeds a threshold. If DaysAnyAdm is less than or equal to this threshold, processing engine 404 initially assigns the individual to the Recovery Guidance intervention group in step 138. Otherwise, the processing engine 404 initially assigns the individual to the Prevention intervention group in step 140.

Referring to FIG. 3C, in step 142, the processing engine 404 determines whether the diagnostic parameter Days Since Inpatient Admission (DaysAnyAdm), which is the number of days since any hospital admission, exceeds a threshold. If DaysAnyAdm is less than or equal to this threshold, processing engine 404 initially assigns the individual to the Recovery Guidance intervention group in step 144. Otherwise, the processing engine 404 proceeds to step 146.

In step 146, the processing engine 404 determines whether the diagnostic parameter Count of Conditions that are Both Chronic and Significant Over the Last 12 Months (Count12mChronicSign), which is a count of significant chronic conditions over the past 12 months, based on Disease Staging, exceeds a threshold. If Count12mChronicSign is greater than this threshold, processing engine 404 initially assigns the individual to the Coordination intervention group in step 148. Otherwise, the processing engine 404 proceeds to step 150.

In step 150, the processing engine 404 determines whether the diagnostic parameter Days Since Last Major Emergency Room Visit (DaysMajorER), which is the number of days since the last major ER visit, exceeds a threshold. If DaysMajorER is less than or equal to this threshold, processing engine 404 initially assigns the individual to the Monitoring intervention group in step 152. Otherwise, the processing engine 404 proceeds to step 154.

In step 154, the processing engine 404 determines whether the diagnostic parameter Count of Unique Intermediate Therapeutic Drug Classes With at Least One Chronic Drug Over the Past 6 Months (CountTherClassChronic6m), which is a count of unique chronic therapeutic classes over the past 6 months, exceeds a threshold. If CountTherClassChronic6m is less than or equal to this threshold, processing engine 404 initially assigns the individual to the Support intervention group in step 156. Otherwise the processing engine 404 initially assigns the individual to the Coordination intervention group in step 158.

Once an initial intervention group is assigned, the algorithm then proceeds to determine whether the individual will be assigned to that initial intervention group or whether an exception exists for assigning the individual to one of the Surveillance, Rebalancing, or Treatment Navigation intervention groups as illustrated in FIG. 3D. The exception rules processing begins in step 180 where the processing engine 404 determines whether the exception rules for the Surveillance intervention group dictate that the individual should be assigned to the Surveillance intervention group. These rules will be discussed in detail below. If it is determined in step 180 that the individual should be assigned to the Surveillance intervention group, the processing engine proceeds to step 182 to check whether the initial intervention group assigned to the individual is a higher priority than the Surveillance intervention group (e.g., the Crisis Management intervention group (see FIG. 1). If the initial intervention group assigned to the individual is not a higher priority than the Surveillance intervention group, the individual is assigned to the Surveillance intervention group in step 184. Otherwise, the individual is assigned to their initial intervention group in step 186.

If the processing engine 404 determines in step 180 that the exception rules for the Surveillance intervention group do not apply, it proceeds to step 188 to then check whether the exception rules for the Rebalancing intervention group dictate that the individual should be assigned to the Rebalancing intervention group. These rules will be discussed in detail below. If it is determined in step 188 that the individual should be assigned to the Rebalancing intervention group, the processing engine proceeds to step 190 to check whether the initial intervention group assigned to the individual is a higher priority than the Rebalancing intervention group (e.g., the Crisis Management intervention group (see FIG. 1). If the initial intervention group assigned to the individual is not a higher priority than the Rebalancing intervention group, the individual is assigned to the Rebalancing intervention group in step 192. Otherwise, the individual is assigned to their initial intervention group in step 186.

If the processing engine 404 determines in step 188 that the exception rules for the Rebalancing intervention group do not apply, it proceeds to step 194 to then check whether the exception rules for the Treatment Navigation intervention group dictate that the individual should be assigned to the Treatment Navigation intervention group. These rules will be discussed in detail below. If it is determined in step 194 that the individual should be assigned to the Treatment Navigation intervention group, the processing engine proceeds to step 196 to check whether the initial intervention group assigned to the individual is a higher priority than the Treatment Navigation intervention group. If the initial intervention group assigned to the individual is not a higher priority than the Treatment Navigation intervention group, the individual is assigned to the Treatment Navigation intervention group in step 198. Otherwise, the individual is assigned to their initial intervention group in step 186.

Referring back to step 180 (FIG. 3D), the processing engine 404 determines whether the individual should be assigned to the Surveillance intervention group based on whether any of the following surveillance exception rules apply:
(1) Can be based on any cancer being actively treated with chemotherapy, radiation therapy, brachytherapy, or related surgery
   (a) Occurs in the most recent 3 months
   (b) Requires a disease category for cancer and at least one of the following:
      (i) Active chemotherapy (Chemo active) in last 3 months (e.g. Capecitabine)
      (ii) Chronic chemotherapy (Chemo chronic) that has not been given prior to the last 3 months (e.g. Tamoxifen)
      (iii) Procedure group that indicates active cancer treatment (Active cancer procgrp) (e.g. Radical resection of tumor)
(2) Can be based on specific cancer type (defined by disease category) and specific procedure groups related to that specific cancer type, occurring in most recent 3 months (e.g. Colectomy for DS Category of Neoplasm, Malignant: Colon and Rectum)
(3) Can be based on DRG indicating major organ transplant
   (a) Occurs in the most recent 3 months
   (b) Hospital admission with qualifying DRG (e.g. DRG 6: Liver Transplant)
(4) Dialysis

Referring to step 188, the processing engine 404 determines whether the individual should be assigned to the Rebalancing intervention group based on whether any of the following rebalancing exception rules apply:
(1) Can be based on new condition:
   (a) First time seen in last year
   (b) Occurs in the most recent 3 months
   (c) Based on qualifying new disease category (e.g. DS Category for Crohn's Disease), new ICD9/ICD10 code (e.g. ICD9 codes 3420-3449 for paralysis), or new MS-DRG (e.g. 61-66 for Stroke)
      i. For asthma, diabetes, and multiple sclerosis patients—must not be on drug for those conditions prior to the most recent 3 months
(2) Can be based on a condition without restriction to new:
   (a) Occurs in the most recent 3 months
   (b) Several ways to qualify:
      (i) Disease category and Stage indicating acute myocardial infarction
      (ii) Psychiatric disease category and place of service of emergency room
      (iii) MS-DRGs related to tracheotomy, amputation, acute myocardial infarction, psychiatric issues, and substance abuse
      (iv) Specific procedure groups containing cpt and HCPCS codes related to amputation, bariatric surgery, colostomy/ileostomy, tracheostomy, tracheotomy

Referring to step 194, the processing engine 404 determines whether the individual should be assigned to the Treatment Navigation intervention group based on whether any of the following treatment navigation exception rules apply:
(1) Based on new condition
   (a) First time seen in last year
   (b) Occurs in most recent month
   (c) In disease category for patient preference sensitive conditions like intervertebral disc disorders of the lumbar spine or benign prostatic hypertrophy

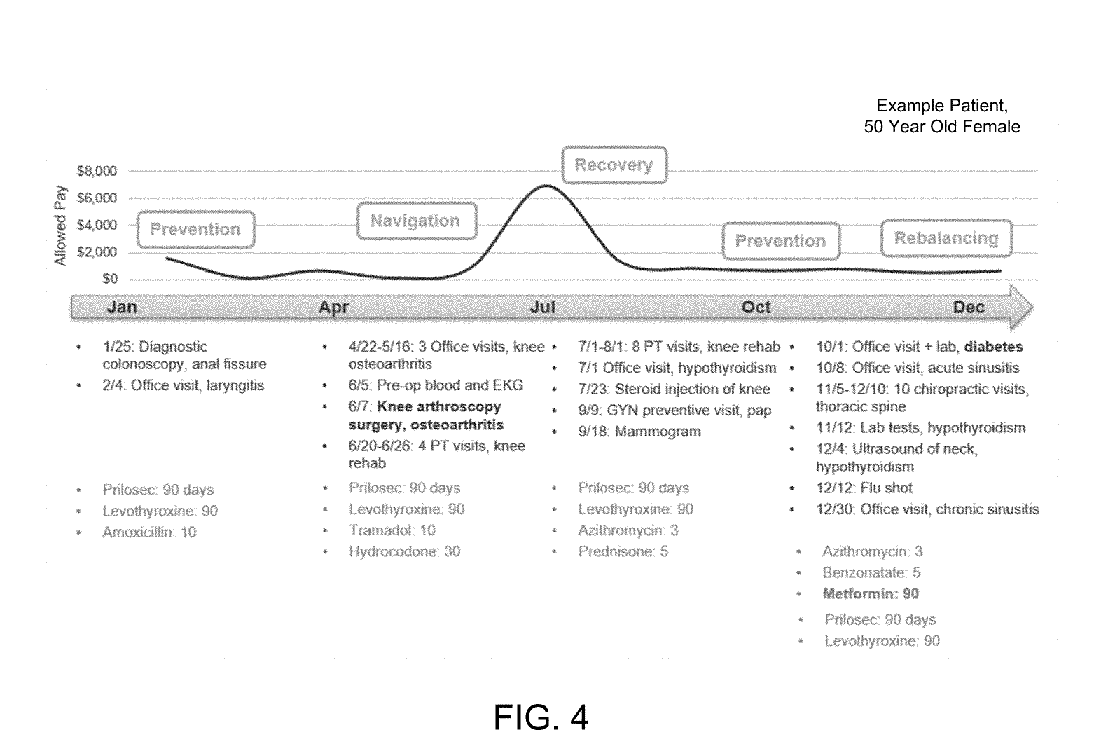

As noted above, the method described herein is used to establish an intervention group for each individual at any given instance in time. However, the intervention group assignment for a individual may change over time. Thus, the algorithm is intended to be re-executed on a periodic basis to identify and account for such changes. FIG. 4 illustrates an example of how an assignment of an intervention group may change over time for an individual.

In the example shown, the intervention groups for a female individual who is age 50, is shown over a one-year period. The individual starts in a Prevention intervention group, changes to a Treatment Navigation intervention group due to a diagnosis of knee osteoarthritis and then a Recovery Guidance intervention group following knee surgery before returning to a Prevention intervention group. The individual is then diagnosed with diabetes and is moved into the Rebalancing intervention group.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in FIGS. 2-3D illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of population classification to identify the likely level of care management required for individuals, for use in a population health management system, the method comprising:
receiving, by a processing engine, medical claims data for an individual;
reviewing, by the processing engine, a plurality of predetermined parameters within the medical claims data for the individual;
classifying, by the processing engine, the individual into one of a plurality of predetermined intervention groups with similar expected care management needs, based on reviewing the plurality of predetermined parameters; and
providing to the population health management system, by the processing engine, a care management level for the individual based on the predetermined intervention group into which the individual has been classified.

2. The method of claim 1, wherein the plurality of predetermined parameters comprises any one or more of the following parameters: a number of days since any hospital admission; a number of hospital admissions for an individual over the last 3 months; a count of significant chronic conditions over the past 12 months based on Disease Staging; a number of chronic stage 3 or higher conditions in past 6 months based on Disease Staging; a number of ER visits for chronic conditions in the past 6 months; a number of days since last major ER visit where major is defined by Disease Staging; a count of office visits over the past 12 months; a number of day's supply of drugs over the past 3 months; a number of day's supply of opiates over the past 3 months; and a count of unique intermediate therapeutic classes over the past 6 months containing at least one chronic drug taken by the individual based on NDC code.

3. The method of claim 1, wherein the plurality of predetermined parameters comprises any one or more of the following parameters: a number of days since any hospital admission; a count of significant chronic conditions over the past 12 months based on Disease Staging; a number of chronic stage 3 or higher claims in past 6 month based on Disease Staging; a count of office visits over the past 12 months; and a number of day's supply of opiates over the past 3 months.

4. The method of claim 1, wherein the plurality of predetermined parameters comprises any one or more of the following parameters: a count of significant chronic conditions over the past 12 months based on Disease Staging; a count of office visits over the past 12 months; and a number of day's supply of opiates over the past 3 months.

5. The method of claim 1, wherein the plurality of predetermined parameters comprises a count of significant chronic conditions over the past 12 months based on Disease Staging.

6. The method of claim 1, wherein the plurality of predetermined intervention groups comprises any one or more of the following intervention groups: Engagement; Prevention; Support; Treatment Navigation; Coordination; Monitoring; Recovery Guidance; Rebalancing; Surveillance; and Crisis Management.

7. The method of claim 1, wherein the plurality of predetermined intervention groups comprises any one or more of the following intervention groups: Engagement; Prevention; Support; Coordination; Monitoring; Recovery Guidance; and Crisis Management.

8. The method of claim 7, wherein after classifying, by the processing engine, the individual into one of a plurality of predetermined intervention groups, based on reviewing the plurality of predetermined parameters, executing exceptions rules to determine if the individual should be classified into one of the following intervention groups: Treatment Navigation; Rebalancing; and Surveillance.

9. The method of claim 7, wherein the plurality of predetermined parameters comprises any one or more of the following parameters: a number of days since any hospital admission; a number of hospital admissions for an individual over the last 3 months; a count of significant chronic conditions over the past 12 months based on Disease Staging; a number of chronic stage 3 or higher conditions in past 6 months based on Disease Staging; a number of ER visits for chronic conditions in the past 6 months; a number of days since last major ER visit; a count of office visits over the past 12 months; a number of day's supply of drugs over the past 3 months; a number of day's supply of opiates over the past 3 months; and a count of unique intermediate therapeutic classes over the past 6 months containing at least one chronic drug taken by the individual based on NDC.

10. A system for population classification to identify likely levels of care management appropriate for individuals, for use in a population health management system, the system comprising:
a processing engine configured to:
receive medical claims data for an individual
review a plurality of predetermined parameters within the medical claims data for the individual;
classify the individual into one of a plurality of predetermined intervention groups, based on reviewing the plurality of predetermined parameters; and
provide to the population health management system, a category indicating the level of care management for the individual based on the predetermined intervention group in which the individual has been classified.

11. The system of claim 10, wherein the plurality of predetermined parameters comprises any one or more of the following parameters: a number of days since any hospital admission; a number of hospital admissions for an individual over the last 3 months; a count of significant chronic conditions over the past 12 months based on Disease Staging; a number of chronic stage 3 or higher claims in past 6 months based on Disease Staging; a number of ER visits for chronic conditions in the past 6 months; a number of days since last major ER visit; a count of office visits over the past 12 months; a number of day's supply of drugs over the past 3 months; a number of day's supply of opiates over the past 3 months; and a count of unique intermediate therapeutic classes over the past 6 months containing at least one chronic drug taken by the individual based on NDC.

12. The system of claim 10, wherein the plurality of predetermined parameters comprises any one or more of the following parameters: a count of significant chronic conditions over the past 12 months based on Disease Staging; a count of office visits over the past 12 months; and a number of day's supply of opiates over the past 3 months.

13. The system of claim 10, wherein the plurality of predetermined intervention groups comprises any one or more of the following intervention groups: Engagement; Prevention; Support; Treatment Navigation; Coordination; Monitoring; Recovery Guidance; Rebalancing; Surveillance; and Crisis Management.

14. The system of claim 10, wherein the plurality of predetermined intervention groups comprises any one or more of the following intervention groups: Engagement; Prevention; Support; Coordination; Monitoring; Recovery Guidance; and Crisis Management.

15. The system of claim 14, wherein after classifying, by the processing engine, the individual into one of a plurality of predetermined intervention groups, based on reviewing the plurality of predetermined parameters, executing exceptions rules to determine if the individual should be classified into one of the following intervention groups: Treatment Navigation; Rebalancing; and Surveillance.

16. A computer program product for population classification to identify care management interventions for individuals, for use in a population health management system. The computer program product comprising a computer readable storage medium having program instructions embodied therewith, the program instructions executable by a processing engine to cause the processing engine to:
receive medical claims data for an individual;
review a plurality of predetermined parameters within the medical claims data for the individual;
classify the individual into one of a plurality of predetermined intervention groups, based on reviewing the plurality of predetermined parameters; and
provide to the population health management system, a recommended level of care management appropriate for the individual based upon the intervention group in which the individual has been classified.

17. The computer program product of claim 16, wherein the plurality of predetermined parameters comprises any one or more of the following parameters: a number of days since any hospital admission; a number of hospital admissions for an individual over the last 3 months; a count of significant chronic conditions over the past 12 months based on Disease Staging; a number of chronic stage 3 or higher claims in past 6 months based on Disease Staging; a number of ER visits for chronic conditions in the past 6 months; a number of days since last major ER visit; a count of office visits over the past 12 months; a number of day's supply of drugs over the past 3 months; a number of day's supply of opiates over the past 3 months; and a count of unique intermediate therapeutic classes over the past 6 months containing at least one chronic drug taken by the individual based on NDC.

18. The computer program product of claim 16, wherein the plurality of predetermined parameters comprises any one or more of the following parameters: a count of significant chronic conditions over the past 12 months based on Disease Staging; a count of office visits over the past 12 months; and a number of day's supply of opiates over the past 3 months.

19. The computer program product of claim 16, wherein the plurality of predetermined intervention groups comprises any one or more of the following intervention groups: Engagement; Prevention; Support; Coordination; Monitoring; Recovery Guidance; and Crisis Management.

20. The computer program product of claim 19, wherein after classifying, by the processing engine, the individual into one of a plurality of predetermined intervention groups, based on reviewing the plurality of predetermined parameters, executing exceptions rules to determine if the individual should be classified into one of the following intervention groups: Treatment Navigation; Rebalancing; and Surveillance.
